(19) United States
(12) Patent Application Publication
(10) Pub. No.: US 2025/0295673 A1
(43) Pub. Date: Sep. 25, 2025

(54) TREATMENT OF BREAST CANCER

(71) Applicant: Edward Lichten, Wellington, FL (US)

(72) Inventor: Edward Lichten, Wellington, FL (US)

(21) Appl. No.: 18/615,197

(22) Filed: Mar. 25, 2024

(51) Int. Cl. A61K 31/58; A61K 9/10; A61K 31/566; A61K 38/095; A61K 47/10; A61K 47/44; A61P 29/00

(52) U.S. Cl. CPC A61K 31/58; A61K 9/10; A61K 31/566; A61K 38/095; A61K 47/10; A61K 47/44; A61P 29/00

(57) ABSTRACT

The present invention provides a composition and method for preventing a cytokine storm from attacking a breast gene, which generally comprises a mixture of anabolic steroids and the administration of same, along with oxandrolone and/or danazol, optionally, oxytocin. The first anabolic steroid used is stanozolol and is used in conjunction with nandralone. The composition is preferably deployed an as injectable liquid suspension and the oxandrolone and oxytocin, where used, are orally ingested. It is also possible to deploy the steroids used herein as an oral administration of the stanozolol along with an injection of nandralone weekly or as a daily topical nandralone cream.

### FIELD OF THE INVENTION

The present invention pertains to means and methods for preventing inflammatory cytokines from activating a breast gene. More particularly, the present invention concerns compositions and methods for preventing inflammatory cytokines from activating a breast gene. Even more particularly, the present invention concerns injectable, topically applied, or partially orally ingested compositions for arresting or abating inflammation of the breast gene.

### PRIOR ART

As is known to those skilled in the art to which the present invention pertains, there are four types of receptors in a breast, namely, alpha receptor, beta receptor, androgen and the progesterone receptor all of which are hormonal receptors. These receptors control the release of cytokines.

Cytokines are a broad group of signaling proteins that are produced transiently, after cellular activation, and act as hormonal regulators which modulate the functions of individual cells. The cytokines regulate processes taking place under normal, developmental and pathological conditions under ordinary circumstances. There exists a belief that what is known as an inflammatory response of cytokines or interleukins occurs where the body starts to attack its own cells and tissues rather than just fighting off a virus. Cytokine-type inflammatory responses, which are inflammatory attacks, are known to occur in autoimmune diseases or conditions foreign bio-active substances. Similarly, such responses may also occur after some types of immune therapy, which can be life threatening and also lead to multiple organ failure.

With respect to breast cancer, it is theorized that if you reduce the number of pathways or sites where cytokines may attach to the breast gene and, thus, the number of cytokine gene interlinkages, per se, there is the possibility of reducing inflammatory responses and possibly tumor formation by precluding activation of the cancer cells. In other words, the breast cancer receptors are blocked.

Thus, the present invention is predicated on the theory that by plugging the receptors with certain steroidal compositions, both anabolic and/or naturally occurring, along with, if necessary, any third steroidal composition, namely, oxandrolone as well as a possible fourth steroidal composition, namely, danazol that the estrogen receptors can be locked. The inclusion of external or internally generated neuropeptide, e.g. oxytocin, further enhances the efficacy of the present invention.

For a more complete understanding of the present invention reference is made to the following detailed description.

### SUMMARY OF THE INVENTION

The present invention provides, in a first aspect, a composition and method for treating breast cancer which, generally, comprises administering an anti-inflammatory mixture of anabolic steroids, optionally and sequentially coupled with a third steroid, namely, oxandrolone, as well as potentially, a fourth steroid, danazol and, optionally therewith, a neuropeptide, namely, oxytocin to prevent cytokine activation of a breast gene.

In another aspect hereof, the steroids are applied using an in vitro inflammatory protocol wherein the steroidal compounds are topically applied as vaginal creams which stimulate an increase in oxytocin in the body without the use of an external source for oxytocin.

For a more complete understanding of the present invention, reference is made to the following detailed description.

### DETAILED DESCRIPTION OF THE INVENTION

At the outset, it is to be noted that there are a number of medications that have different effectiveness to interrupt activation of the breast gene by the inflammatory cytokines. Although not wishing to be bound by any theory, according to the in vitro inflammatory protocol, the more androgens and bioavailable testosterone that is made available to a patient, renders the inflammatory response, i.e., less inflammatory response means less gene activation.

Testosterone and even more so, nandrolone and/or oxandrolone, as well as the combination thereof (these androgens being hormones), bind the loci in the estrogen receptor-alpha and beta 10 to 30 times greater in potency than estradiol. Oxandrolone functions the same as testosterone. Thus, oxandrolone and nandrolone bind strongly to both estrogen and androgen receptors. Stanozolol releases all three of these androgenic anabolic steroids from SHBG so they provide a net increase of androgens by four-fold. Further, stanozolol, when topically applied vaginally, increases brain oxytocin. Higher brain oxytocin levels reduce brain and body inflammation. Oxytocin, by an unknown action, blocks inflammation in the body and brain.

Accordingly, and in accordance with a first embodiment of the present invention, there is provided a method of “plugging” the estrogen receptors in the breasts through the use of a combination of anti-inflammatory anabolic and/or naturally occurring steroids and optionally, a third steroid and, optionally, the neuropeptide oxytocin. It is also possible to use a fourth steroidal compound danazol.

The first and second steroids are a combination of nandralone, which is a naturally occurring steroid and the androgen steroid stanozolol which is a synthetic steroid. The third steroid is synthesized oxandrolone as is the fourth steroid danazol. The nandralone and stanozolol may be administered intravenously as an injectable liquid suspension in amounts ranging from about 20 mg to about 40 mg of nandralone per ml of liquid and from about 15 mg to about 30 mg of stanozolol per 1 ml of the liquid used from the suspension.

When the composition is deployed an as injectable liquid suspension, the steroidal composition is prepared by adding the stanozolol and nandralone suspensions together.

Similarly, the stanozolol may be orally ingested and the nandralone may be either injected or used in a topical cream.

Stanozolol is a commercially available product either as a tablet or as a capsule with dosages ranging from about 2 ml to about 5 ml per dosage and is sold under the commercial name Winstrol.

Nandralone, as is known to the skilled artisan, is a naturally occurring steroid usually sold as a decanoate ester under the name Deca-Durabolin. The less common phenylpropionate ester is also commercially available and sold under the name Durabolin. Nandralone is a white crystalline powder, which forms an oily, yellow suspension when suspended in benzyl alcohol and suitable oil, such as peanut oil, grapeseed oil, sesame oil and the like.

As noted above, in order to enhance the efficacy of the use of nandrolone and stanozolol, a second anabolic steroid, namely, oxandrolone may be used.

Oxandrolone is an anabolic steroid which is commercially available under the names Oxandrin and Anavar. It is orally ingested. In the practice of the present invention, it is taken in a dosage of about 2.5 g. Generally, about 100 mg of the oxandrolone is taken on a weekly basis. Where danazol is used, it is administered as a 100 to 200 mg oral tablet taken 3 times per day or may be compounded into a 2-4% topical cream.

As noted, in practicing the present invention, the first anabolic steroid used is preferably stanozolol which is used in conjunction with a second anabolic steroid, namely, oxandrolone.

It is also possible to deploy the steroids used herein as an oral administration of the stanozolol, alone, or with the oxandrolone along with an injection of nandralone weekly or daily and either orally or as a topical cream. Stanozolol is ordinarily available as either a tablet or a capsule in anywhere from a two to 20 mg dosages. It is also commercially available as a topical cream.

Where the combination of oral ingestion of stanozolol plus the injection of nandralone is used, generally about 20 to about 50 mg per week of stanozolol is ingested, along with an injection of nandrolone in the amount of about 20 mg to about 40 mg of nandrolone in one ml of suspension. The nandralone is administered as a single injection on a weekly basis. The stanozolol is taken over about one week in the requisite amounts to achieve an about 20 to an about 50 mg per week dosage of suspension or cream.

As noted above, a requisite amount of oxytocin may also be used in combination with the steroids if the natural oxytocin levels caused by inflammation do not achieve the desired levels. Oxytocin may be administered either as a vaginal cream, as a tablet or as an injection, ordinarily in amounts ranging from about 50 to about 250 units on a weekly basis.

It is also possible to administer stanozolol separately from the nandralone as a topical vaginal cream which, in and of itself, raises a user's oxytocin levels.

It should be noted that the use of nandrolone, alone, also suppresses the FSH (Follicle Stimulating Hormone) and LH (Leutinizing Hormone) release from the pituitary. The present treatment focuses on plugging up the androgen-receptor (A-R) receptor on the endometriotic cell walls. Nandrolone has an affinity to stick tightly into the A-R; about three times stronger than testosterone and about ten to thirty times stronger than any of the estrogens, i.e. estrone, estradiol, xeno-estrogen. When nandrolone is in the receptor, the breast walls dry up.

In order to test the efficacy of the present invention, a series of tests are administered to breast cancer survivors over a desirable period of time.

The tests are begun by those breast cancer patients who have inflammation in their systems, as diagnosed, using in vitro inflammation protocol and separating the triple negative breast cancer patients into those who found drugs to reduce inflammation from those patients who did not evidence anti-inflammatory findings.

It was observed that those patients having inflammation in their system who were not administered anti-inflammatory drugs died much sooner than those who were administered the anti-inflammatory steroids defined herein. Those who were administered the anti-inflammatory drugs did not have an observable increase in inflammation after one year of observation.

The tests were administered using vaginal topical creams along with the supplemental oxytocin and oxandrolone sequentially to determine the anti-inflammatory efficacy in patients having breast cancer. The tests generally were conducted using MRI, CTR, mammography, blood tests and the like.

It was determined that the inflammation abated over the period of time which the testing was done.

Likewise, if the tests had shown that inflammation had increased, then the use of oxandrolone would be indicated. If inflammation was contra-indicated, then the protocol was to be continued.

1. A method for preventing an inflammation attack on a breast gene, comprising:
administering an effective amount of a mixture of anabolic steroids consisting of stanozolol and nandralone.

2. The method of claim 1 wherein:
each of the stanozolol and nandralone steroids is administered as a suspension thereof.

3. The method of claim 2 wherein:
(a) the nandralone is suspended in benzyl alcohol and an oil selected from the group consisting of peanut oil, grape seed oil and sesame oil; and
(b) the stanzolol is suspended in a mixture of paraffin water, benzyl alcohol and sesame oil.

4. The method of claim 3 wherein:
the mixture of steroid suspensions is administered as an injection.

5. The method of claim 4 wherein:
the suspension comprises:
from about 0.5:1 to about 2.0:1 weight ratio of nandralone to stanozolol, the suspension containing from about 20 mg to about 40 mg of nandralone per one ml of liquid and from about 15 mg to about 30 mg of stanozolol per one ml of liquid.

6. The method of claim 4 wherein:
the injections are administered over a period of seven to fourteen days and from once to twice weekly.

7. The method of claim 1 wherein:
the stanozolol is used any combination with oxandrolone are each administered orally as a tablet and the nandralone is administered as an injection, the oxandrolone tablet comprising at least 2.5 gm thereof.

8. The method of claim 7 wherein:
on a weekly basis from about 20 mg to about 50 mg of stanozolol is orally ingested and from about 20 mg to about 40 mg of nandralone in a one ml suspension of benzyl alcohol and an oil selected from the group consisting of peanut oil, grape seed oil and sesame oil is injected as a liquid suspension once per week and the oxandrolone is administered daily.

9. The method of claim 1, which further comprises administering an effective amount of oxytocin.

10. The method of claim 9 wherein the external oxytocin is administered as a topical vaginal cream.

11. The method of claim 1 wherein the stanzolol and nandralone are administered as a vaginal cream.
